(19) United States
(12) Patent Application Publication
(10) Pub. No.: US 2026/0021156 A1
(43) Pub. Date: Jan. 22, 2026

(54) A METHOD FOR TREATING INFLAMMATION

(71) Applicant: MOLEAC PTE LTD., Singapore (SG)

(72) Inventors: Michel Maurice Jacques LAZDUNSKI, Nice (FR); Hervé DE CELLERY D’ALLEN, Antony (FR); Joris Boris Nicolas LEREDDE, Le Cannet (FR); Catherine Louise Claude HEURTEAUX, La Colle-Sur-Loup (FR)

(21) Appl. No.: 19/112,005

(22) PCT Filed: Sep. 15, 2023
(86) PCT No.: PCT/SG2023/050629

§ 371 Date: Mar. 14, 2025

Related U.S. Application Data
(60) Provisional application No. 63/407,054, filed on Sep. 15, 2022.

(51) Int. Cl. A61K 36/736; A61K 36/232; A61K 36/236; A61K 36/286; A61K 36/481; A61K 36/537; A61K 36/65; A61K 36/69; A61K 36/882; A61P 25/00; A61P 29/00

(52) U.S. Cl. CPC A61K 36/736; A61K 36/232; A61K 36/236; A61K 36/286; A61K 36/481; A61K 36/537; A61K 36/65; A61K 36/69; A61K 36/882; A61P 25/00; A61P 29/00

(57) ABSTRACT

The present disclosure relates to a method of treating neuroinflammation. The disclosure, in one aspect. provides for the administration of an effective amount of at least the four herbal components Polygalae (thin leaf milkwort), Astragali (membranous milkvetch), Ligusticum chuanxiong and Angelica senensis (Chinese angelica), their roots or rhizomes, or extracts thereof to a subject with neuroinflammation.

### TECHNICAL FIELD

The present disclosure relates to a method of treating, controlling, preventing and/or reducing the risk of inflammation of the central nervous system (CNS) by administering to a subject a herbal composition comprising a combination of traditional Chinese herbal medicinal products or extracts thereof.

### BACKGROUND

Neuroinflammation is an inflammation of the nervous tissue, such as the brain and spinal cord. Neuroinflammation is a response of the innate immune system of the CNS that is triggered following an insult caused, for example, by injury, infection, exposure to a toxin, neurodegenerative disease, aging (e.g. “inflammaging”), or resulting from a chemical, mechanical or surgical intervention to treat an underlying condition or disease.

Neuroinflammation can be mediated by the production of reactive oxygen species, chemokines and cytokines. These mediators are produced by CNS glia, including microglia and astrocytes, as well as endothelial cells and peripherally derived immune cells. Acting similarly to macrophages during systemic inflammation, microglia cells are thought to be an important cell type involved in neuroinflammation. In response to an insult, microglia have the ability to shift into different functional states, modifying their proliferation, morphology (i.e., shortened processes), phagocytic activity (clearing foreign organisms and cellular debris) and antigen presentation. However, in addition to these beneficial processes in response to an insult, microglia cells also release pro-inflammatory factors such as cytokines, chemokines and matrix metalloproteinases (MMPs) which can compromise the function of the blood-brain-barrier (BBB). Overall, the multiphasic (beneficial and detrimental) role of neuroinflammation is time dependent, but prolonged activation of microglia causes damage to brain tissue and to the BBB.

There is a lack of effective and safe therapeutic agents that can modulate neuroinflammation to limit or prevent the negative effects of neuroinflammation on brain tissue and the BBB. New therapeutic methods to modulate neuroinflammation are therefore urgently needed.

### SUMMARY

The present disclosure provides a combination of traditional Chinese herbal medicinal products or extracts thereof, where the combination reduces microglia activation and the secretion of inflammatory cytokines and other mediators of neuroinflammation following an insult. The combination is therefore particularly effective at preventing and/or controlling neuroinflammation, treating, reducing or preventing symptoms associated with excessive activation of microglia, and mitigating immune, physiological, biochemical, and psychological consequences of neuroinflammation, including damage to brain tissue and to the BBB.

Thus, according to a first aspect, there is provided a method of treating neuroinflammation in a subject in need thereof, comprising administering to the subject an effective amount of a herbal composition comprising at least the four herbal components Polygalae (thin leaf milkwort), Astragali (membranous milkvetch), Ligusticum chuanxiong and Angelica senensis (Chinese angelica), their roots or rhizomes, or extracts thereof. As used in this aspect, and following aspects and embodiments, “treating” has the meaning defined in the subsequent section entitled “GLOSSARY OF TERMS” unless it is specifically stated hereinafter to have a different meaning.

In one embodiment of the above aspect, a herbal composition comprising the four herbal components Polygalae (thin leaf milkwort), Astragali (membranous milkvetch), Ligusticum chuanxiong and Angelica senensis (Chinese angelica), their roots or rhizomes, or extracts thereof, further comprises at least one herbal component selected from the group consisting of Salviae miltiorrhizae (red sage), Paeoniae rubra (red peony), flower of Carthamus tinctorius (safflower), Semen persicae (Prunus persica seed) and Acori tatarinowii (grassleaf sweetflag), their roots or rhizomes, or an extract thereof.

In a further aspect, there is provided a method of treating neuroinflammation in a subject in need thereof, comprising administering to the subject an effective amount of a herbal composition consisting essentially of at least the four herbal components Polygalae (thin leaf milkwort), Astragali (membranous milkvetch), Ligusticum chuanxiong, Angelica senensis (Chinese angelica), their roots or rhizomes, or extracts thereof.

In another aspect, there is provided a method of treating neuroinflammation in a subject in need thereof, comprising administering to the subject an effective amount of a herbal composition consisting essentially of at least the nine herbal components Polygalae (thin leaf milkwort), Astragali (membranous milkvetch), Ligusticum chuanxiong, Angelica senensis (Chinese angelica), Salviae miltiorrhizae (red sage), Paeoniae rubra (red peony), flower of Carthamus tinctorius (safflower), Semen persicae (Prunus persica seed) and Acori tatarinowii (grassleaf sweetflag), their roots or rhizomes, or extracts thereof.

In one embodiment of any of the above aspects, the herbal composition is a pharmaceutical composition. The pharmaceutical composition may also comprise one or more pharmaceutically acceptable carriers or excipients.

In one embodiment of any of the above aspects, the neuroinflammation is associated with an injury, such as spinal cord injury, stroke, traumatic brain injury, subarachnoid hemorrhage, subdural hemorrhage or extradural hemorrhage.

In one embodiment of any of the above aspects, the neuroinflammation is associated with an infection, such as an infection of the brain or central nervous system (e.g. encephalitis or bacterial, viral or fungal meningitis), or a severe general infection such as sepsis or abscess. Examples of viral infections include Herpes Zoster Virus (VZV) infection, Herpes Simplex Virus (HSV-1 or HSV-2), SARS-COV-2 virus or a variant thereof, Epstein-Barr virus, Hepatitis B virus, Hepatitis C virus, Human immunodeficiency virus type 1 (HIV-1), Human papilloma virus (HPV), Human T-cell lymphotropic virus type I (HTLV-1) and Kaposi sarcoma herpesvirus (KSHV). Examples fungi that can lead to neuroinflammation include Cryptococcus neoformans, Candida albicans, Histoplasma capsulanon, Coccidioides inmitis, Paracoccidioides brasiliensis, Aspergillus spp., and zygomycetes. Examples of bacteria that can lead to neuroinflammation include Listeria monocytogenes, Borrelia burgdorferi, Neisseria meningitidis, Streptococcus pneumoniae, Staphylococcus aureus, Haemophilus influenzae, Mycobacterium and Brucella species. Neuroinflammation may also be associated with a parasitic infection.

In one embodiment of any of the above aspects, the neuroinflammation is associated with an exposure to a toxin.

In one embodiment of any of the above aspects, the neuroinflammation is associated with a neurodegenerative disease, such as Alzheimer's disease, Parkinson's disease, Huntington's disease, frontotemporal dementia, amyotrophic lateral sclerosis, multiple sclerosis, 10-12 primary tauopathies, synucleinopathies (i.e., Lewy Body Dementia and multisystem atrophy), polyglutamine diseases including spinocerebellar ataxias, prion disease, traumatic brain injury, chronic traumatic encephalopathy, stroke and spinal cord injury.

In one embodiment of any of the above aspects, the neuroinflammation is associated with a neuropsychiatric condition, such as depression, stress, post-traumatic stress disorder, schizophrenia, autism spectrum disorder or epilepsy.

In one embodiment of any of the above aspects, the neuroinflammation is associated with aging (e.g. “inflammaging”).

Other disorders that may lead to neuroinflammation include, for example, post-operative cognitive dysfunction, ischemia, brain tumor, chronic pain, headache, migraine, trigeminal neuralgia, cardiovascular disease and associated risk factors (e.g. due to obesity, hypertension, diabetes, hyperlipidemia, smoking etc.), a chronic peripheral inflammatory condition (e.g. rheumatoid arthritis, inflammatory bowel disease, psoriasis etc.), gut microbiota perturbation (i.e. dysbiosis) and herpes keratitis.

In one embodiment of any of the above aspects, the neuroinflammation is associated with a chemical intervention to treat an underlying condition or disease. One example of such a chemical intervention is the use of a TNF inhibitor to treat an autoimmune disease such as rheumatoid arthritis.

In one embodiment of any of the above aspects, the neuroinflammation is associated with a mechanical intervention to treat an underlying condition or disease.

In one embodiment of any of the above aspects, the neuroinflammation is associated with a surgical intervention to treat an underlying condition or disease. Examples of surgical interventions include carotid endarterectomy, carotid angioplasty with or without stenting, vertebral artery angioplasty with or without stenting, intracranial arterial angioplasty with or without stenting, aneurysm coiling, obliteration of vascular malformations, repair of intracranial fistula, thrombectomy, extracranial-intracranial bypass. open heart surgery, cardiac surgery requiring bypass machine, coronary bypass grafting, coronary angioplasty with or without stenting, valvular surgery/valvuloplasty, valvulotomy, closure of patent foramen ovale and surgery of great vessels of the heart.

In one embodiment of any of the above aspects, there is provided the administration of an effective amount of the herbal composition which is from about 1 mg/kg to about 100 mg/kg.

In one embodiment of any of the above aspects, there is provided a method that comprises administering, with an aforementioned herbal composition, a second agent to treat an underlying condition or disease.

In one embodiment of any of the above aspects, there is provided the administration of an effective amount of the herbal composition to control neuroinflammation to facilitate recovery after a brain lesion.

In one embodiment of any of the above aspects, there is provided the administration of an effective amount of the herbal composition to protect the brain against the damaging effects of neuroinflammation and facilitate recovery after a brain lesion.

In one embodiment of any of the above aspects, there is provided the administration of an effective amount of the herbal composition to modulate the inflammatory cascade to favor neurovascular repair.

In one embodiment of any of the above aspects, there is provided the administration of an effective amount of the herbal composition to provide a barrier against the damaging effects of neuroinflammation to facilitate recovery after a brain lesion.

In one embodiment of any of the above aspects, there is provided the administration of an effective amount of the herbal composition to reinforce the blood-brain barrier against the damaging effects of neuroinflammation and facilitate recovery after a brain lesion.

In a particular embodiment of any of the above aspects and embodiments, the neuroinflammation is treated or mitigated in a subject with an underlying condition. In one specific embodiment, the underlying condition is diabetes mellitus.

### BRIEF DESCRIPTION OF THE DRAWINGS

The accompanying drawings are included to illustrate specific embodiments of the present disclosure and the related art. The drawings provide a further understanding of the present disclosure, and are incorporated in, and constitute a part of, the present disclosure. It should be appreciated that the drawings illustrate implementations of the disclosure and, together with the rest of the disclosure, explain the principles of the disclosure. It should be apparent that the drawings exemplify embodiments of the present disclosure, and a person having ordinary skill in the art may readily appreciate other embodiments from the drawings described herein.

FIG. 1 shows the chronology of the experimental protocol for an in vitro inflammation study.

FIG. 2 shows the chronology of the experimental protocol for an in vivo study.

FIG. 3 is a table showing the sequences of qPCR (quantitative polymerase chain reaction) primers used.

FIG. 4 shows the effect of MLC1501 (defined hereinafter) on the relative expression of interleukin-1β (IL-1β), interleukin-6 (IL-6), tumor necrosis factor-alpha (TNF-α), NOD-, LRR- and pyrin domain-containing protein 3 (NLRP3) and inducible nitric oxide synthase (iNOS) genes after stimulation of immortalized mouse microglia cell line (BV2) cells with lipopolysaccharide (LPS).

FIG. 5 shows the dose-response effect of MLC1501 on the LPS-induced increase in the relative expression of IL-1β, IL-6 and TNF-α on BV2 cells.

FIG. 6 shows the effect of MLC1501 on the relative expression of IL-1β and IL-6 following middle cerebral artery occlusion (MCAO)-induced cerebral ischemia.

### GLOSSARY OF TERMS

This section is intended to provide guidance on the interpretation of the words and phrases set forth below (and where appropriate grammatical variants thereof).

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about” as used in relation to a numerical value means, for example, about ±30%, about ±20%, about ±10%, about ±5%, or about ±1% of the numerical value. Where necessary, the word “about” may be omitted from the definition of the disclosure herein.

The words “a”, “an” and “the” are employed to describe elements and components of the invention. This is done merely for convenience and to give a general sense of the invention. This description should be read to include one or at least one and the singular also includes the plural, unless the context clearly indicates otherwise. Thus, for example, the term “an agent” includes a reference to a single agent as well as a plurality of agents (including mixtures of agents). It should also be noted that the term “or” is generally employed in its sense including “and/or” unless the content clearly dictates otherwise.

The term “inflammaging” as used herein means an age-related increase in pro-inflammatory markers in blood and/or tissue which can contribute to the pathogenesis of age-related diseases.

The term “in vivo” as used herein includes a reference to using a whole, living organism. This contrasts with the term “in vitro” where a whole, living organism is not used. The term “in vitro” is to be understood as including, inter alia, “ex vivo” uses whereby cells, tissue etc. which does not form part of a whole, living organism may be employed (e.g. cells or tissues from cell or tissue cultures, biopsies, dead organisms etc.). Further non-limiting examples of “in vitro” relate to the use of cellular extracts or lysates.

The terms “patient” and “subject” are used interchangeably herein and the terms include a reference to any human or non-human animal (preferably a mammal) that it is desired to treat using the present invention. However, it will be understood that “patient” or “subject” does not imply that symptoms are present. The term “mammal” as used herein refers to any member of the class Mammalia, including, without limitation, humans and non-human primates such as chimpanzees and other apes and monkey species; farm animals such as cattle, sheep, pigs, goats and horses; domestic/companion animals such as dogs and cats; laboratory animals (e.g. rabbits and rodents such as mice, rats, and guinea pigs, and the like). Preferably, the mammal is human.

The terms “treatment” and “treating” include any use which remedies a disease state or symptoms, prevents the establishment of disease, or otherwise prevents, hinders, retards, or reverses the progression of disease or other undesirable symptoms in any way whatsoever. Hence, “treatment” and “treating” include prophylactic and therapeutic treatment. In the context herein of “a method of treating neuroinflammation”, the “treating” encompasses modulating neuroinflammation to limit or prevent the negative effects of neuroinflammation on brain tissue and the BBB, while having limited impact on the beneficial effects thereof.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The terms “disease”, “disorder” and “condition” may be used herein interchangeably, unless the context clearly dictates otherwise.

Unless otherwise indicated, all technical and scientific terms used herein have the same meaning as commonly understood to one of ordinary skill in the art to which the invention belongs.

### DETAILED DESCRIPTION

Detailed embodiments and implementations of the claimed subject matters are disclosed herein in detail with the technical matters, structural features, achieved objects, and effects with reference to the accompanying drawings as follows. It shall be understood that the disclosed embodiments and implementations are merely illustrative of the claimed subject matters which may be embodied in various forms. The present disclosure may, however, be embodied in many different forms and should not be construed as limited to the exemplary embodiments and implementations set forth herein. Rather, these exemplary embodiments and implementations are provided so that description of the present disclosure is thorough and complete and will fully convey the scope of the present disclosure to those skilled in the art. Specifically, the terminologies in the embodiments of the present disclosure are merely for describing the purpose of the certain embodiment, but not to limit the disclosure. In the description below, details of well-known features and techniques may be omitted to avoid unnecessarily obscuring the presented embodiments and implementations.

The configurations discussed in the following description are non-limiting examples that can be varied and are cited merely to illustrate at least one embodiment and are not intended to limit the scope thereof.

Microglia are the main resident macrophages of the central nervous system and represent 5-12% of brain cells. Microglia act as cell effectors and thus have an important role in the immune response of the central nervous system. Microglia is therefore crucial in the body's defense and tissue repair processes under physiological conditions. However, the pro-inflammatory immune response driven by microglia is also a key contributor to the pathogenesis of several brain diseases. Exacerbated activation of microglia, e.g., by LPS or by ischemia, can induce a deleterious inflammatory response by leading to the release of chemokines and proinflammatory cytokines such as IL-1β, IL-6, and TNF-α. This release of cytokines and chemokines can occur through various intermediaries.

One key step in the inflammatory response is the activation of inflammasomes, which are intracellular protein complexes comprising pattern recognition receptors and other inflammatory molecules. It is known that the secretion of IL-1β is directly mediated by the formation of inflammasomes, including NLRP3.

Another mediator of proinflammatory cytokine and chemokine secretion is iNOS. It can be induced by both endotoxins (such as LPS) and cytokines. Its induction leads to the production of Nitric Oxide (NO), a free radical, associated with the cytotoxic function of microglia, apoptosis and oxidative stress.

These cytokines and proinflammatory mediators, in addition to leading to neuronal death, facilitate damage to the blood-brain barrier and thus promote leukocyte infiltration into the brain.

Certain herbal compositions are identified herein which limit the release of proinflammatory cytokines, including the cytokines IL-1β, IL-6, and TNF-α. The herbal compositions herein also limit the formation of inflammasomes, including NLRP3 and iNOS. The herbal compositions are therefore particularly useful for the treatment of inflammation where these cytokines and inflammasomes are implicated, including the treatment of neuroinflammation.

Thus, in one aspect of the present disclosure, there is provided a method of treating neuroinflammation in a subject in need thereof, comprising administering to the subject an effective amount of a herbal composition comprising at least the four herbal components Radix Polygalae (root of thin leaf milkwort), Radix Astragali (root of membranous milkvetch), Rhizome Ligusticum chuanxiong and Radix Angelica senensis (root of Chinese angelica) or extracts thereof.

In one embodiment of the above aspect, the herbal composition comprises the four herbal components Radix Polygalae (root of thin leaf milkwort), Radix Astragali (root of membranous milkvetch), Rhizome Ligusticum chuanxiong and Radix Angelica senensis (root of Chinese angelica) and further comprises at least one herbal component selected from the group consisting of Radix et Rhizome Salviae miltiorrhizae (red sage root), Radix Paeoniae rubra (red peony root), flower of Carthamus tinctorius (safflower), Semen persicae (Prunus persica seed) and Rhizome Acori tatarinowii (rhizome of grassleaf sweetflag) or an extract thereof.

In a further aspect, there is provided a method of treating neuroinflammation in a subject in need thereof, comprising administering to the subject an effective amount of a herbal composition consisting essentially of at least the four herbal components Radix Polygalae (root of thin leaf milkwort), Radix Astragali (root of membranous milkvetch), Rhizome Ligusticum chuanxiong and Radix Angelica senensis (root of Chinese angelica) or extracts thereof.

In another aspect, there is provided a method of treating neuroinflammation in a subject in need thereof, comprising administering to the subject an effective amount of a herbal composition consisting essentially of at least the nine herbal components Radix Polygalae (root of thin leaf milkwort), Radix Astragali (root of membranous milkvetch), Rhizome Ligusticum chuanxiong and Radix Angelica senensis (root of Chinese angelica), Radix et Rhizome Salviae miltiorrhizae (red sage root), Radix Paeoniae rubra (red peony root), flower of Carthamus tinctorius (safflower), Semen persicae (Prunus persica seed) and Rhizome Acori tatarinowii (rhizome of grassleaf sweetflag) or extracts thereof.

In one embodiment of any of the above aspects, the neuroinflammation is treated or mitigated in a subject with an underlying condition, such as a cardiovascular disease, diabetes, obesity or atherosclerosis. In one specific embodiment, the subject with an underlying condition, such as a cardiovascular disease, diabetes, obesity or atherosclerosis is treated with a herbal composition as described above to reduce the risk of damaging effects to the brain caused by neuroinflammation. In one specific embodiment, the underlying condition is diabetes mellitus. In one specific embodiment, the diabetic subject is treated with a herbal composition as described above to improve post-stroke recovery following an ischemic stroke.

In one embodiment of any of the above aspects, the herbal composition is a pharmaceutical composition. The pharmaceutical composition may also comprise one or more pharmaceutically acceptable carriers or excipients.

In one embodiment of any of the above aspects, the herbal composition comprises extracts of the aforementioned herbal components.

In one embodiment of any of the above aspects, the herbal composition comprises 5 times the weight-to-weight ratio of Radix Astragali or an extract thereof compared to the weight of each of the other herbal components present or extracts thereof.

In one embodiment of any of the above aspects, the pharmaceutical composition is MLC901.

In one embodiment of any of the above aspects, the pharmaceutical composition is MLC1501.

In one embodiment of any of the above aspects, the neuroinflammation is the result of an insult, such as an injury, for example, an injury selected from a spinal cord injury, stroke, traumatic brain injury, subarachnoid hemorrhage, subdural hemorrhage or extradural hemorrhage.

In a particular embodiment of any of the above aspects, the neuroinflammation arises following an ischemic stroke. In this embodiment, treatment of the subject with a herbal composition described herein, including treatment with MLC901, or treatment with MLC1501, provides:
1. An increase in the rate of functional independence/autonomy recovery after about 3 months to about 24 months following first administration of the herbal composition. In one aspect, the functional independence/autonomy recovery results from controlling, modulating, protecting, and/or reinforcing the blood-brain barrier against the damaging effects of neuroinflammation;
2. A reduction in the time to achieve functional independence/autonomy recovery by about 18 months. In one aspect, the functional independence/autonomy recovery results from controlling, modulating, protecting, and/or reinforcing the blood-brain barrier against the damaging effects of neuroinflammation;
3. An acceleration in achieving functional independence/autonomy recovery. In one aspect, the functional independence/autonomy recovery results from controlling, modulating, protecting, and/or reinforcing the blood-brain barrier against the damaging effects of neuroinflammation;
4. A decrease in the duration of hospitalization. In one aspect, the decrease in the time spend in a hospital is a result of controlling, modulating, protecting, and/or reinforcing the blood-brain barrier against the damaging effects of neuroinflammation;
5. A reduction of the time and/or cost associated with recovery of functional independence/autonomy. In one aspect, the functional independence/autonomy recovery results from controlling, modulating, protecting, and/or reinforcing the blood-brain barrier against the damaging effects of neuroinflammation; and/or
6. An improvement in the quality of life. In one aspect, the improvement results from controlling, modulating, protecting, and/or reinforcing the blood-brain barrier against the damaging effects of neuroinflammation.

In a particular embodiment of any of the above aspects, the neuroinflammation arises following a trauma-triggered neurodegenerative disease, such as a traumatic brain injury (TBI) or a spinal cord injury. In this embodiment, treatment of the subject with a herbal composition described herein, including treatment with MLC901, or treatment with MLC1501, provides a reduction in the severity of post-injury symptoms. In a specific embodiment, reduction in the severity of post-injury symptoms is achieved by controlling, modulating, protecting, and/or reinforcing the blood-brain barrier against the damaging effects of neuroinflammation. Following a TBI, treatment of the subject with a herbal composition described herein, including treatment with MLC901, or treatment with MLC1501, may also provide an improvement in recovery of cognitive domains, such as complex attention and/or executive functioning, and/or may have a positive effect on symptoms of anxiety or depression and/or on quality of life. In a specific embodiment, recovery of cognitive domains is achieved by controlling, modulating, protecting, and/or reinforcing the blood-brain barrier against the damaging effects of neuroinflammation.

In a particular embodiment of any of the above aspects, treatment of the subject with a herbal composition described herein, including treatment with MLC901, or treatment with MLC1501, improves a mood disorder associated with a neurodegenerative disease. In a specific embodiment, improvement in the mood disorder is achieved by controlling, modulating, protecting, and/or reinforcing the blood-brain barrier against the damaging effects of neuroinflammation.

In a particular embodiment of any of the above aspects, the neuroinflammation arises due to the subject suffering from dementia or Alzheimer's Disease. In this embodiment, treatment of the subject with a herbal composition described herein, including treatment with MLC901, or treatment with MLC1501, provides:
1. An improvement in recovery of cognitive functions (e.g. memory, praxis and/or language) within about 3 months to about 48 months following first administration of the herbal composition. In one aspect, the improvement in recovery results from controlling, modulating, protecting, and/or reinforcing the blood-brain barrier against the damaging effects of neuroinflammation;
2. A slowing down of disease progression. In one aspect, the slowing down of disease progression results from controlling, modulating, protecting, and/or reinforcing the blood-brain barrier against the damaging effects of neuroinflammation; and/or
3. A disease-modifying effect. In one aspect, the disease-modifying effect results from controlling, modulating, protecting, and/or reinforcing the blood-brain barrier against the damaging effects of neuroinflammation.

In one embodiment of any of the above aspects, the neuroinflammation is the result of an insult, such as an infection, for example, an infection of the brain or central nervous system (e.g. encephalitis or bacterial, viral or fungal meningitis), or a severe infection such as sepsis or abscess. Examples of viral infections include Herpes Zoster Virus (VZV) infection, Herpes Simplex Virus (HSV-1 or HSV-2), SARS-COV-2 virus or a variant thereof, Epstein-Barr virus, Hepatitis B virus, Hepatitis C virus, Human immunodeficiency virus type 1 (HIV-1), Human papilloma virus (HPV), Human T-cell lymphotropic virus type I (HTLV-1) and Kaposi sarcoma herpesvirus (KSHV). Examples fungi that can lead to neuroinflammation include Cryptococcus neoformans, Candida albicans, Histoplasma capsulatum, Coccidioides immitis, Paracoccidioides brasiliensis, Aspergillus spp., and zygomycetes. Examples of bacteria that can lead to neuroinflammation include Listeria monocytogenes, Borrelia burgdorferi, Neisseria meningitidis, Streptococcus pneumoniae. Staphylococcus aureus, Haemophilus influenzae, Mycobacterium and Brucella species. Neuroinflammation may also be associated with a parasitic infection.

In a specific embodiment, the neuroinflammation arises due to systemic inflammation from the peripheral immune system of a subject caused by an infection such as COVID or “long COVID”. In this embodiment, treatment of the subject with a herbal composition described herein, including treatment with MLC901, or treatment with MLC1501, provides protection against progression of neurodegenerative processes by controlling, modulating, protecting, and/or reinforcing the blood-brain barrier against the damaging effects of neuroinflammation.

In one embodiment of any of the above aspects, the neuroinflammation is the result of exposure to a toxin.

In one embodiment of any of the above aspects, the neuroinflammation is associated with a neurodegenerative disease, such as Alzheimer's disease, Parkinson's disease, Huntington's disease, frontotemporal dementia, amyotrophic lateral sclerosis, multiple sclerosis, 10-12 primary tauopathies, synucleinopathies (i.e., Lewy Body Dementia and multisystem atrophy), polyglutamine diseases including spinocerebellar ataxias, prion disease, traumatic brain injury, chronic traumatic encephalopathy, stroke and spinal cord injury.

In one embodiment, the neuroinflammation is associated with a neuropsychiatric condition, such as depression, stress, post-traumatic stress disorder, schizophrenia, autism spectrum disorder or epilepsy.

In one embodiment of any of the above aspects, the neuroinflammation is associated with aging (e.g. “inflammaging”).

Other disorders that may lead to neuroinflammation include post-operative cognitive dysfunction, ischemia, brain tumor, chronic pain, headache, migraine, trigeminal neuralgia, cardiovascular disease and associated risk factors (e.g. due to obesity, hypertension, diabetes, hyperlipidemia, smoking etc.), a chronic peripheral inflammatory condition (e.g. rheumatoid arthritis, inflammatory bowel disease, psoriasis etc.), gut microbiota perturbation (i.e. dysbiosis) and herpes keratitis.

In one embodiment, the neuroinflammation arises due to systemic inflammation from the peripheral immune system of a subject caused by gut microbiota perturbation (i.e. dysbiosis). In this embodiment, treatment of the subject with a herbal composition described herein, including treatment with MLC901, or treatment with MLC1501, provides protection against progression of neurodegenerative processes by controlling, modulating, protecting, and/or reinforcing the blood-brain barrier against the damaging effects of neuroinflammation.

In one embodiment of any of the above aspects, the neuroinflammation results from a chemical intervention to treat an underlying condition or disease. One example of such a chemical intervention is the use of a TNF inhibitor to treat an autoimmune disease such as rheumatoid arthritis.

In one embodiment of any of the above aspects, the neuroinflammation results from a mechanical intervention to treat an underlying condition or disease.

In one embodiment of any of the above aspects, the neuroinflammation results from a surgical intervention to treat an underlying condition or disease. Examples of surgical interventions include carotid endarterectomy, carotid angioplasty with or without stenting, vertebral artery angioplasty with or without stenting, intracranial arterial angioplasty with or without stenting, aneurysm coiling, obliteration of vascular malformations, repair of intracranial fistula, thrombectomy, extracranial-intracranial bypass. open heart surgery, cardiac surgery requiring bypass machine, coronary bypass grafting, coronary angioplasty with or without stenting, valvular surgery/valvuloplasty, valvulotomy, closure of patent foramen ovale and surgery of great vessels of the heart.

In one embodiment of any of the above aspects, an effective amount of the herbal composition to treat neuroinflammation is from about 1 mg/kg to about 100 mg/kg.

In one embodiment of any of the above aspects, there is provided a method that comprises administering, in addition to a herbal composition herein to treat neuroinflammation, a second agent to treat an underlying condition or disease.

In one embodiment of any of the above aspects, the subject is diagnosed as having neuroinflammation, such as by a healthcare professional.

In one embodiment of any of the above aspects, the subject is tested for neuroinflammation by measuring the levels of pro-inflammatory chemokines and/or cytokines, such as the levels of one or more cytokines selected from IL.-1β, IL-6 and TNF-α.

In one embodiment of any of the above aspects, the subject is tested for neuroinflammation by measuring the levels of inflammasomes, including NLRP3 and/or iNOS.

In one aspect, a subject confirmed as having neuroinflammation following testing (e.g. by identifying that the subject exhibits elevated levels of pro-inflammatory chemokines and/or cytokines, such as elevated levels of one or more cytokines selected from IL-1β, IL-6 and TNF-α and and/or elevated levels of one or more inflammasomes such as NLRP3 and/or iNOS) is treated with an effective amount of a herbal composition comprising at least the four herbal components Radix Polygalae (root of thin leaf milkwort), Radix Astragali (root of membranous milkvetch), Rhizome Ligusticum chuanxiong and Radix Angelica senensis (root of Chinese angelica) or extracts thereof.

In a further aspect, a subject confirmed as having neuroinflammation following testing (e.g. by identifying that the subject exhibits elevated levels of pro-inflammatory chemokines and/or cytokines, such as elevated levels of one or more cytokines selected from IL-1β, IL-6 and TNF-α and and/or elevated levels of one or more inflammasomes such as NLRP3 and/or iNOS) is treated with an effective amount of a herbal composition comprising the four herbal components Radix Polygalae (root of thin leaf milkwort), Radix Astragali (root of membranous milkvetch), Rhizome Ligusticum chuanxiong and Radix Angelica senensis (root of Chinese angelica) or extracts thereof together with at least one further herbal component selected from the group consisting of Radix et Rhizome Salviae miltiorrhizae (red sage root), Radix Paeoniae rubra (red peony root), flower of Carthamus tinctorius (safflower), Semen persicae (Prunus persica seed) and Rhizome Acori tatarinowii (rhizome of grassleaf sweetflag) or an extract thereof.

In another aspect, a subject confirmed as having neuroinflammation following testing (e.g. by identifying that the subject exhibits elevated levels of pro-inflammatory chemokines and/or cytokines, such as elevated levels of one or more cytokines selected from IL-1β, IL-6 and TNF-α and and/or elevated levels of one or more inflammasomes such as NLRP3 and/or iNOS) is treated with an effective amount of a herbal composition consisting essentially of at least the four herbal components Radix Polygalae (root of thin leaf milkwort), Radix Astragali (root of membranous milkvetch), Rhizome Ligusticum chuanxiong and Radix Angelica senensis (root of Chinese angelica) or extracts thereof.

In yet another aspect, a subject confirmed as having neuroinflammation following testing (e.g. by identifying that the subject exhibits elevated levels of pro-inflammatory chemokines and/or cytokines, such as elevated levels of one or more cytokines selected from IL-1β, IL-6 and TNF-α and and/or elevated levels of one or more inflammasomes such as NLRP3 and/or iNOS) is treated with an effective amount of a herbal composition consisting essentially of at least the nine herbal components Radix Polygalae (root of thin leaf milkwort), Radix Astragali (root of membranous milkvetch), Rhizome Ligusticum chuanxiong, Radix Angelica senensis (root of Chinese angelica), Radix et Rhizome Salviae miltiorrhizae (red sage root), Radix Paeoniae rubra (red peony root), flower of Carthamus tinctorius (safflower), Semen persicae (Prunus persica seed) and Rhizome Acori tatarinowii (rhizome of grassleaf sweetflag) or extracts thereof.

In one embodiment of any of the above aspects, the herbal composition comprises extracts of the aforementioned herbal components.

In one embodiment of any of the above aspects, the herbal composition comprises 5 times the weight-to-weight ratio of Radix Astragali or an extract thereof compared to the weight of each of the other herbal components present or extracts thereof.

In one embodiment of any of the above aspects, where the subject is confirmed as having neuroinflammation following testing, the herbal composition used to treat the subject is MLC901.

In one embodiment of any of the above aspects, where the subject is confirmed as having neuroinflammation following testing, the herbal composition used to treat the subject is MLC1501.

In one aspect, a subject at risk of neuroinflammation as a result of an insult (e.g. due to an aforementioned condition, disease, injury, infection, age, or chemical, mechanical or surgical intervention) is treated to prevent or limit neuroinflammation by administering (before, at the time of, or subsequent to the insult) an effective amount of a herbal composition comprising at least the four herbal components Radix Polygalae (root of thin leaf milkwort), Radix Astragali (root of membranous milkvetch), Rhizome Ligusticum chuanxiong and Radix Angelica senensis (root of Chinese angelica) or extracts thereof.

In a further aspect, a subject at risk of neuroinflammation as a result of an insult (e.g. due to an aforementioned condition, disease, injury, infection, age, or chemical, mechanical or surgical intervention) is treated to prevent or limit neuroinflammation by administering (before, at the time of, or subsequent to the insult) an effective amount of a herbal composition comprising the four herbal components Radix Polygalae (root of thin leaf milkwort), Radix Astragali (root of membranous milkvetch), Rhizome Ligusticum chuanxiong and Radix Angelica senensis (root of Chinese angelica) or extracts thereof together with at least one further herbal component selected from the group consisting of Radix et Rhizome Salviae miltiorrhizae (red sage root), Radix Paeoniae rubra (red peony root), flower of Carthamus tinctorius (safflower), Semen persicae (Prunus persica seed) and Rhizome Acori tatarinowii (rhizome of grassleaf sweetflag) or an extract thereof.

In another aspect, a subject at risk of neuroinflammation as a result of an insult (e.g. due to an aforementioned condition, disease, injury, infection, age, or chemical, mechanical or surgical intervention) is treated to prevent or limit neuroinflammation by administering (before, at the time of, or subsequent to the insult) an effective amount of a herbal composition consisting essentially of at least the four herbal components Radix Polygalae (root of thin leaf milkwort), Radix Astragali (root of membranous milkvetch), Rhizome Ligusticum chuanxiong and Radix Angelica senensis (root of Chinese angelica) or extracts thereof.

In yet another aspect, a subject at risk of neuroinflammation as a result of an insult (e.g. due to an aforementioned condition, disease, injury, infection, age, or chemical, mechanical or surgical intervention) is treated to prevent or limit neuroinflammation by administering (before, at the time of, or subsequent to the insult) an effective amount of a herbal composition consisting essentially of at least the nine herbal components Radix Polygalae (root of thin leaf milkwort), Radix Astragali (root of membranous milkvetch), Rhizome Ligusticum chuanxiong, Radix Angelica senensis (root of Chinese angelica), Radix et Rhizome Salviae miltiorrhizae (red sage root), Radix Paeoniae rubra (red peony root), flower of Carthamus tinctorius (safflower), Semen persicae (Prunus persica seed) and Rhizome Acori tatarinowii (rhizome of grassleaf sweetflag) or extracts thereof.

In one embodiment of any of the above aspects, the herbal composition comprises extracts of the aforementioned herbal components.

In one embodiment of any of the above aspects, the herbal composition comprises 5 times the weight-to-weight ratio of Radix Astragali or an extract thereof compared to the weight of each of the other herbal components present or extracts thereof.

In one embodiment of any of the above aspects, where the subject is at risk of neuroinflammation as a result of an insult, the herbal composition used to prevent or limit neuroinflammation is MLC901.

In one embodiment of any of the above aspects, where the subject is at risk of neuroinflammation as a result of an insult, the herbal composition used to prevent or limit neuroinflammation is MLC1501.

In one aspect, a subject at risk of developing a disease or condition as a result neuroinflammation is treated with an effective anti-inflammatory amount of a herbal composition comprising at least the four herbal components Radix Polygalae (root of thin leaf milkwort), Radix Astragali (root of membranous milkvetch), Rhizome Ligusticum chuanxiong and Radix Angelica senensis (root of Chinese angelica) or extracts thereof to prevent or mitigate the development of the disease or condition.

In a further aspect, a subject at risk of developing a disease or condition as a result neuroinflammation is treated with an effective anti-inflammatory amount of a herbal composition comprising the four herbal components Radix Polygalae (root of thin leaf milkwort), Radix Astragali (root of membranous milkvetch), Rhizome Ligusticum chuanxiong and Radix Angelica senensis (root of Chinese angelica) or extracts thereof together with at least one further herbal component selected from the group consisting of Radix et Rhizome Salviae miltiorrhizae (red sage root), Radix Paeoniae rubra (red peony root), flower of Carthamus tinctorius (safflower), Semen persicae (Prunus persica seed) and Rhizome Acori tatarinowii (rhizome of grassleaf sweetflag) or an extract thereof to prevent or mitigate the development of the disease or condition.

In another aspect, a subject at risk of developing a disease or condition as a result neuroinflammation is treated with an effective anti-inflammatory amount of a herbal composition consisting essentially of at least the four herbal components Radix Polygalae (root of thin leaf milkwort), Radix Astragali (root of membranous milkvetch), Rhizome Ligusticum chuanxiong and Radix Angelica senensis (root of Chinese angelica) or extracts thereof to prevent or mitigate the development of the disease or condition.

In yet another aspect, a subject at risk of developing a disease or condition as a result neuroinflammation is treated with an effective anti-inflammatory amount of a herbal composition consisting essentially of at least the nine herbal components Radix Polygalae (root of thin leaf milkwort), Radix Astragali (root of membranous milkvetch), Rhizome Ligusticum chuanxiong, Radix Angelica senensis (root of Chinese angelica), Radix et Rhizome Salviae miltiorrhizae (red sage root), Radix Paeoniae rubra (red peony root), flower of Carthamus tinctorius (safflower), Semen persicae (Prunus persica seed) and Rhizome Acori tatarinowii (rhizome of grassleaf sweetflag) or extracts thereof to prevent or mitigate the development of the disease or condition.

In one embodiment of any of the above aspects, the herbal composition comprises extracts of the aforementioned herbal components.

In one embodiment of any of the above aspects, the herbal composition comprises 5 times the weight-to-weight ratio of Radix Astragali or an extract thereof compared to the weight of each of the other herbal components present or extracts thereof.

In one embodiment of any of the above aspects, where the subject is at risk of developing a disease or condition as a result neuroinflammation, the herbal composition used to prevent or mitigate the development of the disease or condition is MLC901.

In one embodiment of any of the above aspects, where the subject is at risk of developing a disease or condition as a result neuroinflammation, the herbal composition used to prevent or mitigate the development of the disease or condition is MLC1501.

The aforementioned “disease or condition” may be, for example, a brain injury, brain swelling, leakage of the blood brain barrier, cerebral stroke, transient ischemic attack, or a neuropsychiatric disorder (e.g. depression, stress, post-traumatic stress disorder, schizophrenia, autism spectrum disorder or epilepsy).

NeuroAid II, referred to herein as “MLC901”, is a composition containing extracts of nine herbal components (Radix Astragali, Radix Salvia miltiorrhizae, Radix Paeoniae rubra, Rhizoma Ligusticum chuanxiong, Radix Angelica senensis, Carthamus tinctorius, Prunus persica, Radix Polygalae and Rhizoma Acori tatarinowii). In MLC901, the Radix Astragali extract is present at 5 times the weight-to-weight ratio of each of the other herbal component extracts present. It is currently marketed as an oral treatment to support post-stroke recovery.

MLC1501 is composition containing extracts of four herbal components (Radix Astragali, Rhizoma Ligusticum chuanxiong, Radix Polygalae and Radix Angelica senensis. In MLC1501, the Radix Astragali extract is present at 5 times the weight-to-weight ratio of each of the other herbal component extracts present. It is currently in human clinical trials to assess efficacy in post-stroke recovery.

MLC901 and MLC1501, and the preparation thereof, are described in published PCT application number WO 2017/048191A1, the contents of which are incorporated by reference herein in their entirety.

In one embodiment of any of the above aspects, the herbal composition of the method herein consists essentially of the four herbal components Radix Polygalae, Radix Astragali, Rhizome Ligusticum chuanxiong and Radix Angelica senensis or extracts thereof, together with any 1, 2, 3, 4 or 5 of the herbal components Radix Salvia miltiorrhizae, Radix Paeoniae rubra, Carthamus tinctorius, Prunus persica, and Rhizoma Acori tatarinowii or extracts thereof. Such herbal compositions, and the preparation thereof, are described in published PCT application numbers WO 2007/106049A1, WO 2010/053456A1, WO 2010/110755A1 and WO 2013/141818A1, the contents of which are incorporated by reference herein in their entirety.

Herbal compositions herein, including MLC901 and MLC1501, are generally very effective in treating or mitigating neuroinflammation in subjects who have had a stroke, especially ischemic stroke, to aid post-stroke recovery. However, patients with an underlying condition, especially where the underlying condition has an inflammatory component, such as in diabetes mellitus, are known to suffer from a poor prognosis of functional recovery following a stroke and standard therapy, including rehabilitation therapy. Stroke in diabetic subjects results in increased mortality as well as long-term neurological and functional disabilities with standard of care treatment. Nevertheless, herbal compositions herein, including MLC901 and MLC1501, have been found to effectively aid post-stroke recovery in diabetic subjects.

Thus, in one aspect of the present disclosure, there is provided a method of treating or mitigating neuroinflammation in a diabetic subject following an ischemic stroke, comprising administering to the subject an effective amount of a herbal composition comprising at least the four herbal components Polygalae (thin leaf milkwort), Astragali (membranous milkvetch), Ligusticum chuanxiong and Angelica senensis (Chinese angelica), their roots or rhizomes, or extracts thereof.

In one embodiment of the above aspect, a herbal composition comprising the four herbal components Polygalae (thin leaf milkwort), Astragali (membranous milkvetch), Ligusticum chuanxiong and Angelica senensis (Chinese angelica), their roots or rhizomes, or extracts thereof, further comprises at least one herbal component selected from the group consisting of Salviae miltiorrhizae (red sage), Paeoniae rubra (red peony), flower of Carthamus tinctorius (safflower), Semen persicae (Prunus persica seed) and Acori tatarinowii (grassleaf sweetflag), their roots or rhizomes, or an extract thereof.

In a further aspect, there is provided a method of treating or mitigating neuroinflammation in a diabetic subject following an ischemic stroke, comprising administering to the subject an effective amount of a herbal composition consisting essentially of four herbal components Polygalae (thin leaf milkwort), Astragali (membranous milkvetch), Ligusticum chuanxiong, Angelica senensis (Chinese angelica), their roots or rhizomes, or extracts thereof.

In another aspect, there is provided a method of treating or mitigating neuroinflammation in a diabetic subject following an ischemic stroke, comprising administering to the subject an effective amount of a herbal composition consisting essentially of nine herbal components Polygalae (thin leaf milkwort), Astragali (membranous milkvetch), Ligusticum chuanxiong, Angelica senensis (Chinese angelica), Salviae miltiorrhizae (red sage), Paeoniae rubra (red peony), flower of Carthamus tinctorius (safflower), Semen persicae (Prunus persica seed) and Acori tatarinowii (grassleaf sweetflag), their roots or rhizomes, or extracts thereof.

In one embodiment of any of the above aspects, the herbal composition is a pharmaceutical composition. The pharmaceutical composition may also comprise one or more pharmaceutically acceptable carriers or excipients. In a specific embodiment, the herbal composition is MLC901. In a specific embodiment, the herbal composition is MLC1501.

In a particular aspect of the present disclosure, there is provided a method of treating or mitigating neuroinflammation in a diabetic subject following an ischemic stroke, comprising administering to the subject an effective amount of MLC901.

In a particular aspect of the present disclosure, there is provided a method of treating or mitigating neuroinflammation in a diabetic subject following an ischemic stroke, comprising administering to the subject an effective amount of MLC1501.

In a particular aspect of the present disclosure, there is provided a method of treating a diabetic subject following an ischemic stroke, comprising administering to the subject an effective amount of MLC901 to improve post-stroke recovery. In one embodiment, the subject is undergoing post-stroke standard of care, such as treatment with an anti-platelet agent (e.g. aspirin) and/or rehabilitation therapy. In one embodiment, diabetic subjects undergoing post-stroke standard of care co-administered MLC901 demonstrate superior recovery rates compared to diabetic subjects who only receive post-stroke standard of care. In one embodiment, superiority is demonstrated within about 3, 6, 9, 12, 18 or 24 months after the stroke occurs.

In a particular aspect of the present disclosure, there is provided a method of treating a diabetic subject following an ischemic stroke, comprising administering to the subject an effective amount of MLC1501 to improve post-stroke recovery. In one embodiment, the subject is undergoing post-stroke standard of care, such as treatment with an anti-platelet agent (e.g. aspirin) and/or rehabilitation therapy. In one embodiment, diabetic subjects undergoing post-stroke standard of care co-administered MLC1501 demonstrate superior recovery rates compared to diabetic subjects who only receive post-stroke standard of care. In one embodiment, superiority is demonstrated within about 3, 6, 9, 12, 18 or 24 months after the stroke occurs.

A pharmaceutical composition herein may optionally comprise one or more pharmaceutically acceptable additives, carriers, and/or diluents. Examples of pharmaceutically acceptable additives include pharmaceutically acceptable excipients, buffers, adjuvants, stabilizers, diluents, fillers, preservatives, lubricants, or other pharmaceutically acceptable materials well known to those skilled in the art or as described herein. Examples of suitable pharmaceutical carriers or diluents include phosphate buffered saline solutions, water, emulsions (such as oil/water emulsions), various types of wetting agents, sterile solutions etc. Examples of excipients which may be employed include, for example, sugars, starches, celluloses, gums, proteins, dextrin and maltodextrin. Various formulations are commonly known and are thoroughly discussed in the latest edition of Remington's Pharmaceutical Sciences (Maack Publishing, Easton PA). In at least some embodiments, a pharmaceutical composition as herein described comprises an excipient, e.g. dextrin or maltodextrin.

In one embodiment of any of the above aspects, a composition of the present disclosure (e.g. a pharmaceutical composition) may be comprised within a kit. The kit may, in addition to the herbal components, comprise instructions for use. The kit may be promoted, distributed, and/or sold as a unit for performing one of the aspects of the present disclosure.

In general, pharmaceutical compositions of the present disclosure may be prepared according to methods known to those of ordinary skill in the art.

The compositions may, for example, be a solution, a suspension, liquid, chopped herbs, powder, a paste, aqueous, non-aqueous or any combination thereof.

The compositions (e.g. pharmaceutical compositions) of the present disclosure may be administered by any suitable route, such as orally, parenterally, intravenously, subcutaneously, intradermally, intraperitoneally or topically, in liquid, semi-liquid or solid form and are formulated in a manner suitable for each route of administration. The term “administering” and variations of that term including “administer” and “administration”, includes contacting, applying, delivering or providing a composition of the present disclosure to an organism, or a surface by any appropriate means.

The herbal components may be administered in a therapeutically effective amount (either as a single dose or as part of a series of doses). By an “effective amount” or a “therapeutically effective amount” is meant the amount administered to achieve physiological significance. An agent is physiologically significant if it is present in an amount that results in a detectable change in the physiology of a recipient patient such that beneficial or desired results are achieved.

The exact amount required will vary from subject to subject depending on factors such as the species being treated, the age, weight and general health of the subject, the condition being treated and the severity of the condition, the mode of administration, the gender of the subject, diet, time and frequency of administration, drug combination(s), and tolerance/response to therapy and so forth.

In one embodiment of any of the above aspects, compositions (e.g. pharmaceutical compositions) of the present disclosure are administered as capsules taken one or more (e.g. 1, 2, 3 or 4) times per day. In a particular embodiment, MLC901 is administered in the form of 2 capsules, taken 3 times per day. In a particular embodiment, MLC1501 is administered in the form of 4 capsules, taken 2 times per day. For patients with swallowing difficulties, capsules may be opened and powder diluted in water that can be drunk as such or injected via a gastric tube.

In one embodiment of any of the above aspects, the patient's daily dose is about 500 mg to about 8 g, for example about 1 g to about 8 g, including about 1 g, 2 g, 3 g, 4 g, 5 g, 6 g, 7 g or 8 g. A “daily dose” can be a single unit dose or multiple unit doses (of tablets, capsules etc.) taken on a given day. However, it is to be understood that the dosage may be varied depending upon the requirement of the patients and the severity of the condition being treated, etc.

The duration of treatment is days or, more typically, months (e.g. three months), adaptable based on the patient's condition. In one embodiment of any of the above aspects, treatment lasts about 12 weeks. In another embodiment of any of the above aspects, treatment lasts about 24 weeks. In another embodiment of any of the above aspects, treatment lasts about 36 weeks. In another embodiment of any of the above aspects, treatment lasts about 48 weeks. In another embodiment of any of the above aspects, treatment lasts longer than about 48 weeks.

The timing of administration of the first dose of the herbal composition of the present disclosure (e.g. MLC901 or MLC1501) should generally occur as soon as possible after an insult caused, for example, by injury, infection, exposure to a toxin, neurodegenerative disease, aging (e.g. “inflammaging”), or resulting from a chemical, mechanical or surgical intervention to treat an underlying condition or disease, that can lead to neuroinflammation. For example, the first dose may be administered within 1, 2, 3, 4, 5, 6, 7, 8, 9 or 10 days (e.g. within 1 to 5 days, 1 to 4 days, 1 to 3 days or 1 to 2 days) following the insult. In one embodiment, the first dose of the herbal composition of the present disclosure (e.g. MLC901 or MLC1501) is administered within 24 hours following the insult, for example within 1, 2, 3, 4, 5, 6, 7, 8, 9, 10, 11 or 12 hours following the insult.

In one embodiment of any of the above aspects, the treatment regimen is designed for acute treatment.

In one embodiment of any of the above aspects, the treatment regimen is designed for chronic treatment.

In one embodiment of any of the above aspects, the treatment regimen is designed for post-acute treatment.

In one embodiment of any of the above aspects, a herbal composition of the present disclosure (e.g. MLC901 or MLC1501) may be administered to a subject in combination with an active agent to treat an underlying condition or disease and/or in combination with additional anti-inflammatory therapy (e.g. one or more anti-inflammatory agents). The one or more further active agents may be administered at the same time as the herbal composition of the present disclosure (e.g. simultaneously) or at different times (e.g. sequentially) and over different periods of time, which may be separate from one another or overlapping.

The one or more further active agents may be administered by the same or different routes from a herbal composition of the present disclosure. The one or more further active agent utilized, and the appropriate administration route and dose level, will be known to those in the art or could be readily determined by one skilled in the art. Typically, as is well known in the medical art, dosage regimens may depend on various factors including the patient's size, body surface area, age, the particular compound to be administered, sex, time and route of administration, general health, and other drugs being administered concurrently. While individual needs vary, determination of optimal ranges of effective amounts of each component is within the skill of the art. Typically, the dosage of the one or more further active agents will be the same or similar to that administered when the agent is used without a herbal composition of the present disclosure. When a herbal composition of the present disclosure is administered with one or more further active agents, the one or more further active agents may be provided in a composition or kit comprising said herbal composition of the present disclosure, or the one or more further active agents may be provided separately (i.e. not as part of the composition or kit providing the herbal composition of the present disclosure).

In addition to the aforementioned embodiments, the present disclosure includes the following specific embodiments:

Embodiment 1: A herbal composition comprising at least the four herbal components Radix Polygalae (root of thin leaf milkwort), Radix Astragali (root of membranous milkvetch), Rhizome Ligusticum chuanxiong and Radix Angelica senensis (root of Chinese angelica) or extracts thereof for use in treating neuroinflammation in a subject.

Embodiment 2: The herbal composition for use according to Embodiment 1, wherein the herbal composition further comprises at least one herbal component selected from the group consisting of Radix et Rhizome Salviae miltiorrhizae (red sage root), Radix Paeoniae rubra (red peony root), flower of Carthamus tinctorius (safflower), Semen persicae (Prunus persica seed) and Rhizome Acori tatarinowii (rhizome of grassleaf sweetflag) or an extract thereof.

Embodiment 3: The herbal composition for use according to Embodiment 1, wherein the herbal composition consists essentially of at least the four herbal components Radix Polygalae (root of thin leaf milkwort), Radix Astragali (root of membranous milkvetch), Rhizome Ligusticum chuanxiong and Radix Angelica senensis (root of Chinese angelica) or extracts thereof.

Embodiment 4: The herbal composition for use according to Embodiment 1, wherein the herbal composition consists essentially of at least the nine herbal components Radix Polygalae (root of thin leaf milkwort), Radix Astragali (root of membranous milkvetch), Rhizome Ligusticum chuanxiong, Radix Angelica senensis (root of Chinese angelica), Radix et Rhizome Salviae miltiorrhizae (red sage root), Radix Paeoniae rubra (red peony root), flower of Carthamus tinctorius (safflower), Semen persicae (Prunus persica seed) and Rhizome Acori tatarinowii (rhizome of grassleaf sweetflag) or extracts thereof.

Embodiment 5: The herbal composition for use according to any one of Embodiments 1 to 4, wherein the herbal composition is a pharmaceutical composition.

Embodiment 6: The herbal composition for use according to Embodiment 5, wherein the pharmaceutical composition also comprises a pharmaceutically acceptable carrier or excipient.

Embodiment 7: The herbal composition for use according to Embodiment 6, wherein the pharmaceutical composition is the composition MLC901.

Embodiment 8: The herbal composition for use according to Embodiment 6, wherein the pharmaceutical composition is the composition MLC1501.

Embodiment 9: The herbal composition for use according to any one of Embodiments 1 to 8, wherein an effective amount of the herbal composition is from about 1 mg/kg to about 100 mg/kg.

Embodiment 10: The herbal composition for use according to any one of Embodiments 1 to 9, wherein the method comprises administering a second anti-inflammatory agent.

Embodiment 11: The herbal composition for use according to any one of Embodiments 1 to 10, wherein the neuroinflammation is the result of an injury.

Embodiment 12: The herbal composition for use according to Embodiment 11, wherein the injury is selected from the group consisting of spinal cord injury, stroke, traumatic brain injury, subarachnoid hemorrhage, subdural hemorrhage or extradural hemorrhage.

Embodiment 13: The herbal composition for use according to any one of Embodiments 1 to 10, wherein the neuroinflammation is the result of an infection.

Embodiment 14: The herbal composition for use according to Embodiment 13, wherein the infection is an infection of the brain or central nervous system (e.g. encephalitis or bacterial, viral or fungal meningitis).

Embodiment 15: The herbal composition for use according to Embodiment 13, wherein the infection is a viral infection [e.g. Herpes Zoster Virus (VZV) infection, Herpes Simplex Virus (HSV-1 or HSV-2), SARS-COV-2 virus or a variant thereof, Epstein-Barr virus, Hepatitis B virus, Hepatitis C virus, Human immunodeficiency virus type 1 (HIV-1), Human papilloma virus (HPV), Human T-cell lymphotropic virus type I (HTLV-1) or Kaposi sarcoma herpesvirus (KSHV)], a fungal infection [e.g. where the fungus is selected from the group consisting of Cryptococcus neoformans, Candida albicans, Histoplasma capsulatum, Coccidioides immitis, Paracoccidioides brasiliensis, Aspergillus spp., and zygomycetes], a bacterial infection [e.g. wherein the bacterium is selected from the group consisting of Listeria monocytogenes, Borrelia burgdorferi, Neisseria meningitidis, Streptococcus pneumoniae, Staphylococcus aureus, Haemophilus influenzae, Mycobacterium and Brucella species] or a parasitic infection

Embodiment 16: The herbal composition for use according to any one of Embodiments 1 to 10, wherein the neuroinflammation is the result of exposure to a toxin.

Embodiment 17: The herbal composition for use according to any one of Embodiments 1 to 10, wherein the neuroinflammation is associated with a neurodegenerative disease [e.g. Alzheimer's disease, Parkinson's disease, Huntington's disease, frontotemporal dementia, amyotrophic lateral sclerosis, multiple sclerosis, 10-12 primary tauopathies, synucleinopathies (i.e., Lewy Body Dementia and multisystem atrophy), polyglutamine diseases including spinocerebellar ataxias, prion disease, traumatic brain injury, chronic traumatic encephalopathy, stroke and spinal cord injury].

Embodiment 18: The herbal composition for use according to any one of Embodiments 1 to 10, wherein the neuroinflammation is associated with a neuropsychiatric condition (e.g. depression, stress, post-traumatic stress disorder, schizophrenia, autism spectrum disorder or epilepsy).

Embodiment 19: The herbal composition for use according to any one of Embodiments 1 to 10, wherein the neuroinflammation is associated with aging (e.g. “inflammaging”).

Embodiment 20: The herbal composition for use according to any one of Embodiments 1 to 10, wherein the neuroinflammation is associated with a disease or disorder selected from a group consisting of post-operative cognitive dysfunction, ischemia, brain tumor, chronic pain, headache, migraine, trigeminal neuralgia, cardiovascular disease and associated risk factors (e.g. due to obesity, hypertension, diabetes, hyperlipidemia, smoking etc.), a chronic peripheral inflammatory condition (e.g. rheumatoid arthritis, inflammatory bowel disease, psoriasis etc.), gut microbiota perturbation (i.e. dysbiosis) and herpes keratitis.

Embodiment 21: The herbal composition for use according to any one of Embodiments 1 to 10, wherein the neuroinflammation results from a chemical intervention to treat an underlying condition or disease. One example of such a chemical intervention is the use of a TNF inhibitor to treat an autoimmune disease such as rheumatoid arthritis.

Embodiment 22: The herbal composition for use according to any one of Embodiments 1 to 10, wherein the neuroinflammation results from a mechanical intervention to treat an underlying condition or disease.

Embodiment 23: The herbal composition for use according to any one of Embodiments 1 to 10, wherein the neuroinflammation results from a surgical intervention to treat an underlying condition or disease.

Embodiment 24: The herbal composition for use according to Embodiment 23, wherein the surgical intervention is selected from the group consisting of carotid endarterectomy, carotid angioplasty with or without stenting, vertebral artery angioplasty with or without stenting, intracranial arterial angioplasty with or without stenting, aneurysm coiling, obliteration of vascular malformations, repair of intracranial fistula, thrombectomy, extracranial-intracranial bypass. open heart surgery, cardiac surgery requiring bypass machine, coronary bypass grafting, coronary angioplasty with or without stenting, valvular surgery/valvuloplasty, valvulotomy, closure of patent foramen ovale and surgery of great vessels of the heart.

Embodiment 25: The herbal composition for use according to any one of Embodiments 21 to 24, wherein the herbal composition is administered prior to, at the same time as, or following the administration of the intervention to treat an underlying condition or disease.

Embodiment 26: The herbal composition for use according to any one of Embodiments 1 to 25, wherein the subject is tested for, and confirmed as having neuroinflammation, prior to administering the herbal composition.

Embodiment 27: The herbal composition for use according to Embodiment 26, wherein the test shows that the subject exhibits elevated levels of pro-inflammatory chemokines and/or cytokines.

Embodiment 28: The herbal composition for use according to Embodiment 26, wherein the test shows that the subject exhibits elevated levels of one or more pro-inflammatory chemokines selected from IL-16, IL-6 and TNF-α and/or elevated levels of one or more inflammasomes selected from NLRP3 and iNOS.

Embodiment 29: A herbal composition comprising at least the four herbal components Radix Polygalae (root of thin leaf milkwort), Radix Astragali (root of membranous milkvetch), Rhizome Ligusticum chuanxiong and Radix Angelica senensis (root of Chinese angelica) or extracts thereof for use in preventing or limiting neuroinflammation in a subject at risk of neuroinflammation as a result of an insult.

Embodiment 30: The herbal composition for use according to Embodiment 29, wherein the herbal composition further comprises at least one herbal component selected from the group consisting of Radix et Rhizome Salviae miltiorrhizae (red sage root), Radix Paeoniae rubra (red peony root), flower of Carthamus tinctorius (safflower), Semen persicae (Prunus persica seed) and Rhizome Acori tatarinowii (rhizome of grassleaf sweetflag) or an extract thereof.

Embodiment 31: The herbal composition for use according to Embodiment 29, wherein the herbal composition consists essentially of at least the four herbal components Radix Polygalae (root of thin leaf milkwort), Radix Astragali (root of membranous milkvetch), Rhizome Ligusticum chuanxiong and Radix Angelica senensis (root of Chinese angelica) or extracts thereof.

Embodiment 32: The herbal composition for use according to Embodiment 29, wherein the herbal composition consists essentially of at least the nine herbal components Radix Polygalae (root of thin leaf milkwort), Radix Astragali (root of membranous milkvetch), Rhizome Ligusticum chuanxiong, Radix Angelica senensis (root of Chinese angelica), Radix et Rhizome Salviae miltiorrhizae (red sage root), Radix Paeoniae rubra (red peony root), flower of Carthamus tinctorius (safflower), Semen persicae (Prunus persica seed) and Rhizome Acori tatarinowii (rhizome of grassleaf sweetflag) or extracts thereof.

Embodiment 33: The herbal composition for use according to any one of Embodiments 29 to 32, wherein the herbal composition is a pharmaceutical composition.

Embodiment 34: The herbal composition for use according to Embodiment 33, wherein the pharmaceutical composition also comprises a pharmaceutically acceptable carrier or excipient.

Embodiment 35: The herbal composition for use according to Embodiment 34, wherein the pharmaceutical composition is the composition MLC901.

Embodiment 36: The herbal composition for use according to Embodiment 34, wherein the pharmaceutical composition is the composition MLC1501.

Embodiment 37: The herbal composition for use according to any one of Embodiments 29 to 36, wherein an effective amount of the herbal composition is from about 1 mg/kg to about 100 mg/kg.

Embodiment 38: The herbal composition for use according to any one of Embodiments 29 to 37, wherein the method comprises administering a second anti-inflammatory agent.

Embodiment 39: The herbal composition for use according to any one of Embodiments 29 to 38, wherein the insult results from a chemical, mechanical or surgical intervention to treat an underlying condition or disease as described in any one of Embodiments 21 to 24.

Embodiment 40: A herbal composition comprising at least the four herbal components Radix Polygalae (root of thin leaf milkwort), Radix Astragali (root of membranous milkvetch), Rhizome Ligusticum chuanxiong and Radix Angelica senensis (root of Chinese angelica) or extracts thereof for use in treating a subject at risk of developing a disease or condition as a result neuroinflammation to prevent or mitigate the development of the disease or condition.

Embodiment 41: The herbal composition for use according to Embodiment 40, wherein the herbal composition further comprises at least one herbal component selected from the group consisting of Radix et Rhizome Salviae miltiorrhizae (red sage root), Radix Paeoniae rubra (red peony root), flower of Carthamus tinctorius (safflower), Semen persicae (Prunus persica seed) and Rhizome Acori tatarinowii (rhizome of grassleaf sweetflag) or an extract thereof.

Embodiment 42: The herbal composition for use according to Embodiment 40, wherein the herbal composition consists essentially of at least the four herbal components Radix Polygalae (root of thin leaf milkwort), Radix Astragali (root of membranous milkvetch), Rhizome Ligusticum chuanxiong and Radix Angelica senensis (root of Chinese angelica) or extracts thereof.

Embodiment 43: The herbal composition for use according to Embodiment 40, wherein the herbal composition consists essentially of at least the nine herbal components Radix Polygalae (root of thin leaf milkwort), Radix Astragali (root of membranous milkvetch), Rhizome Ligusticum chuanxiong, Radix Angelica senensis (root of Chinese angelica), Radix et Rhizome Salviae miltiorrhizae (red sage root), Radix Paeoniae rubra (red peony root), flower of Carthamus tinctorius (safflower), Semen persicae (Prunus persica seed) and Rhizome Acori tatarinowii (rhizome of grassleaf sweetflag) or extracts thereof.

Embodiment 44: The herbal composition for use according to any one of Embodiments 40 to 43, wherein the herbal composition is a pharmaceutical composition.

Embodiment 45: The herbal composition for use according to Embodiment 44, wherein the pharmaceutical composition also comprises a pharmaceutically acceptable carrier or excipient. Embodiment 46: The herbal composition for use according to Embodiment 45, wherein the pharmaceutical composition is the composition MLC901.

Embodiment 47: The herbal composition for use according to Embodiment 45, wherein the pharmaceutical composition is the composition MLC1501.

Embodiment 48: The herbal composition for use according to any one of Embodiments 40 to 47, wherein an effective amount of the herbal composition is from about 1 mg/kg to about 100 mg/kg.

Embodiment 49: The herbal composition for use according to any one of Embodiments 1 to 48, wherein the subject has a brain injury.

Embodiment 50: The herbal composition for use according to any one of Embodiments 1 to 48, wherein the subject has brain swelling.

Embodiment 51: The herbal composition for use according to any one of Embodiments 1 to 48, wherein the subject has leakage of the blood brain barrier.

Embodiment 52: The herbal composition for use according to any one of Embodiments 1 to 48, wherein the subject has cerebral stroke.

Embodiment 53: The herbal composition for use according to any one of Embodiments 1 to 48, wherein the subject has a neuropsychiatric disorder (e.g. depression, stress, post-traumatic stress disorder, schizophrenia, autism spectrum disorder or epilepsy).

Embodiment 54: A herbal composition comprising Radix Polygalae (root of thin leaf milkwort), Radix Astragali (root of membranous milkvetch), Rhizome Ligusticum chuanxiong and Radix Angelica senensis (root of Chinese angelica) or extracts thereof for use in treating neuroinflammation in a subject with diabetes mellitus.

Embodiment 55: A herbal composition comprising Radix Polygalae (root of thin leaf milkwort), Radix Astragali (root of membranous milkvetch), Rhizome Ligusticum chuanxiong, Radix Angelica senensis (root of Chinese angelica), Radix et Rhizome Salviae miltiorrhizae (red sage root), Radix Paeoniae rubra (red peony root), flower of Carthamus tinctorius (safflower), Semen persicae (Prunus persica seed) and Rhizome Acori tatarinowii (rhizome of grassleaf sweetflag) or extracts thereof for use in treating neuroinflammation in a subject with diabetes mellitus.

Embodiment 56: The herbal composition for use according to Embodiment 54, wherein the herbal composition consists essentially of Radix Polygalae (root of thin leaf milkwort), Radix Astragali (root of membranous milkvetch), Rhizome Ligusticum chuanxiong and Radix Angelica senensis (root of Chinese angelica) or extracts thereof.

Embodiment 57: The herbal composition for use according to Embodiment 55, wherein the herbal composition consists essentially of Radix Polygalae (root of thin leaf milkwort), Radix Astragali (root of membranous milkvetch), Rhizome Ligusticum chuanxiong, Radix Angelica senensis (root of Chinese angelica), Radix et Rhizome Salviae miltiorrhizae (red sage root), Radix Paeoniae rubra (red peony root), flower of Carthamus tinctorius (safflower), Semen persicae (Prunus persica seed) and Rhizome Acori tatarinowii (rhizome of grassleaf sweetflag) or extracts thereof.

Embodiment 58: The herbal composition for use according to any one of Embodiments 54 to 57, wherein the herbal composition is a pharmaceutical composition.

Embodiment 59: The herbal composition for use according to Embodiment 58, wherein the pharmaceutical composition also comprises a pharmaceutically acceptable carrier or excipient.

Embodiment 60: The herbal composition for use according to Embodiment 58, wherein the pharmaceutical composition is the composition MLC901.

Embodiment 61: The herbal composition for use according to Embodiment 58, wherein the pharmaceutical composition is the composition MLC1501.

Embodiment 62: The herbal composition for use according to any one of Embodiments 54 to 61, wherein an effective amount of the herbal composition is from about 1 mg/kg to about 100 mg/kg.

Embodiment 63: The herbal composition for use according to any one of Embodiments 54 to 62, wherein the method comprises administering a second anti-inflammatory agent.

Embodiment 64: The herbal composition for use according to any one of Embodiments 54 to 63, wherein the subject is tested for, and confirmed as having neuroinflammation, prior to administering the herbal composition.

Embodiment 65: The herbal composition for use according to Embodiment 64, wherein the test shows that the subject exhibits elevated levels of pro-inflammatory chemokines and/or cytokines.

Embodiment 66: The herbal composition for use according to Embodiment 64, wherein the test shows that the subject exhibits elevated levels of one or more pro-inflammatory chemokines selected from IL-1β, IL-6 and TNF-α and/or elevated levels of one or more inflammasomes selected from NLRP3 and iNOS.

Embodiment 67: The herbal composition for use according to any one of Embodiments 54 to 66, wherein the neuroinflammation is caused by a brain injury.

Embodiment 68: The herbal composition for use according to any one of Embodiments 54 to 66, wherein the neuroinflammation is caused by brain swelling.

Embodiment 69: The herbal composition for use according to any one of Embodiments 54 to 66, wherein the neuroinflammation is caused by leakage of the blood brain barrier.

Embodiment 70: The herbal composition for use according to any one of Embodiments 54 to 66, wherein the neuroinflammation is caused by cerebral stroke.

Embodiment 71: The herbal composition for use according to any one of Embodiments 54 to 66, wherein the neuroinflammation is caused by a neuropsychiatric disorder (e.g. depression, stress, post-traumatic stress disorder, schizophrenia, autism spectrum disorder or epilepsy).

The Examples hereinafter describe a study to investigate the effects of MLC1501 on inflammation, and more specifically its effects on the expression of various cellular markers involved in neuroinflammation. The study was divided into 2 parts:
1/Characterization of the effects of MLC1501 following LPS-induced inflammation, with a focus on the in vitro expression of the markers IL-1β, IL-6, TNF-α, NLRP3 and iNOS.
2/Characterization of the effects of MLC1501 in a model of focal cerebral ischemia, with a focus on the in vivo expression of pro-inflammatory cytokines IL-1β and IL-6.

Results obtained hereinafter for MLC1501 may also be achieved with other herbal compositions of the present disclosure, including MLC901.

### EXAMPLE 1

### 1) Materials & Methods

### a) Cell Culture

The BV-2 microglial cell line was derived from neonatal murine microglia immortalized with v-raf/v-myc oncogenes (Blasi et al., 1990). It was cultured in DMEM “high glucose” medium supplemented with 5% Fetal Calf Serum (FCS) and 1% Penicillin/Streptomycin (Gibco, Thermofisher Scientific, USA). The cells were maintained in a humidified incubator at 37° C. and in an atmosphere containing 5% CO2. As soon as the cells reached 80% confluence, they were used for the experiments.

### b) In Vitro Treatment

The cells were pre-treated for 24 hours with MLC1501 at the concentration of 250 μg/mL. To perform a dose-response analysis, 8 different concentrations of MLC1501 were chosen: 0, 10, 50, 100, 250, 500, 1000 and 2000 μg/mL.

### c) In Vitro Inflammation Model: LPS Stimulation

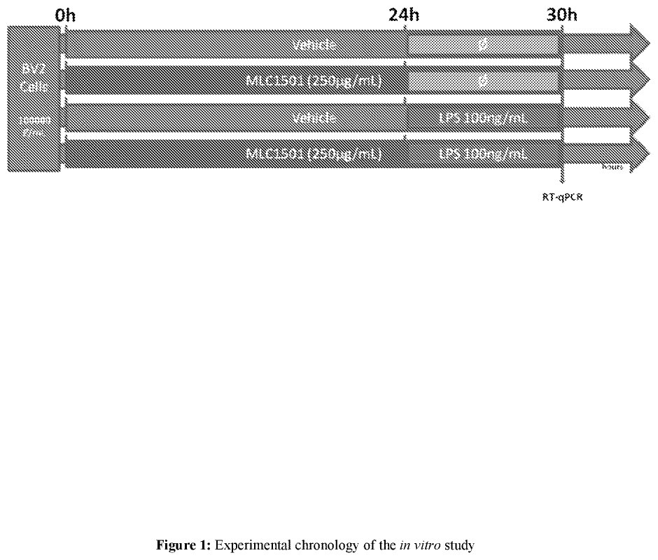

Cells are stimulated with 100 ng/ml of LPS (E. coli; 0111: B4, Sigma-Aldrich) for 6 hours in the absence of SVF but in the presence of MLC1501 (250 μg/mL). The experimental protocol is shown in FIG. 1.

### d) Animals

Male C57B1/6JRj mice aged 7 weeks (at the time of receipt) were kept 5 per cage in an animal house maintained at an ambient temperature of 21±1° C. The animals had access to food and drink ad libitum.

### e) In Vivo Treatment

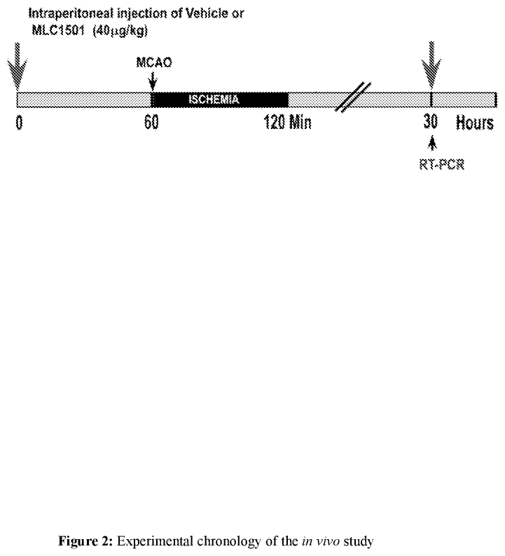

A single 40 μg/kg dose of MLC1501 (100 μL bolus) diluted in saline (vehicle) was intraperitoneally injected 60 minutes before the onset of ischemia. The schematic of the protocol is shown in FIG. 2.

### f) MCAO Model of Ischemic Stroke

A 60-minute focal ischemia model was performed on the vehicle-treated and MLC1501-treated mice. The model consists of the occlusion of the right middle cerebral artery (MCAO) for 60 minutes with a silicone-coated 6-0 filament (Doccol, Redlands, CA, USA), followed by reperfusion (or in some cases without reperfusion). Cerebral blood flow in the region of interest is monitored by laser Doppler (Perimed, Craponne France) to control the severity of the pattern and the presence of reperfusion. Animals with less than 70% reduction in cerebral blood flow are excluded from the study (<1%). A sham operation is performed for a group of control animals (insertion of the filament into the carotid artery without causing occlusion of the MCA). The animals were then housed in a thermostatically controlled recovery chamber (30-31° C.) supplied with synthetic air for 30 hours.

g) mRNA Extraction and Quantitative rt-PCR

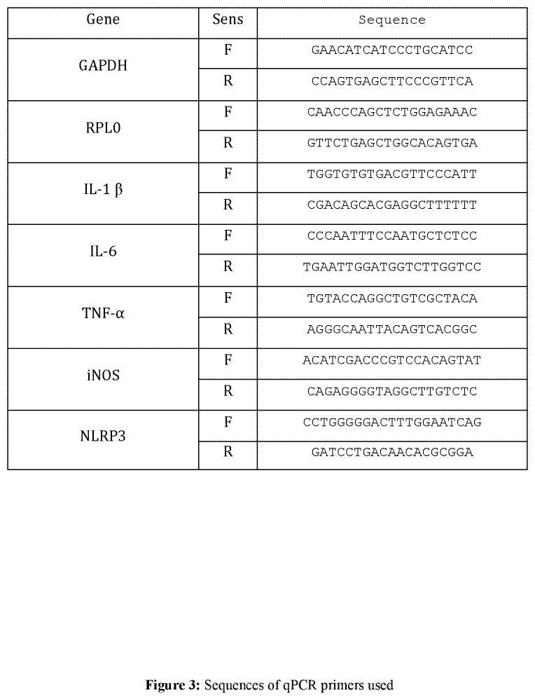

Microglial cells and ipsilateral brain hemispheres were collected 30 h after the start of each in vitro and in vivo protocol. The mRNA from microglial cells and brain tissues were extracted with RNAzol (Euromedex, France), and then reverse-transcribed using Superscript IV (Thermofisher Scientific, USA). Expression of mRNAs encoding IL-1β, IL-6 and TNF-α genes was then determined by qPCR (SybrGreen 480 Roche) using specific primers (Eurogentec, Angers, France) (sequences detailed in FIG. 3). Results were normalized by the expression of a housekeeping gene, GAPDH or RPL0. Values are expressed as 2-ΔΔCT normalized by the controls. A duplicate was performed for each sample.

### h) Statistics and Regressions

Data are presented as Mean±SEM with each sample being the average of 2 replicates. The regressions were done on the GraphPad Prism software. Outliers were determined by the ROUT method (Q=1%). Statistical analyses between the different groups of animals were calculated with a Mann & Whitney test. The level of significance was set at P<0.05.

### 2) Results

### a) MLC1501 Reduces the Relative Expression of Genes Involved in the Pro-Inflammatory Response in the In Vitro Inflammation Model:

1/Characterization of the anti-inflammatory effects of MLC1501 at the concentration of 250 μg/mL on the BV2 microglial cell line following LPS stimulation

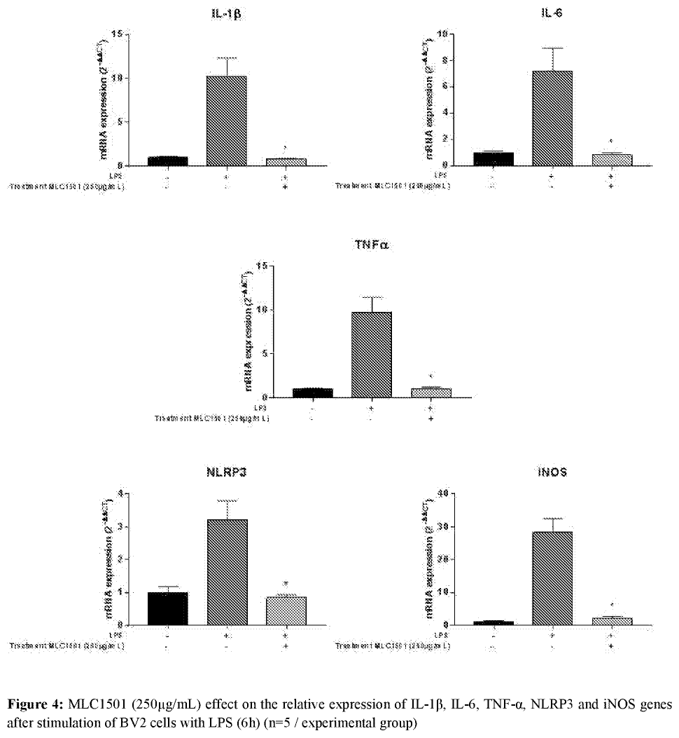

We observed that 6 h of LPS stimulation induced on cells not treated with MLC1501 produced a strong increase in mRNA expression levels encoding pro-inflammatory cytokine genes IL-1β, IL-6 and TNF-α. The same LPS stimulation also induced in untreated cells an increase in the expression of NLRP3 and iNOS genes (FIG. 4). Administration of MLC1501 at a concentration of 250 μg/mL led to a significant reduction in the relative expression of IL-1β, IL-6, TNF-α, NLRP3 and iNOS genes after 6 h of LPS stimulation compared with the vehicle administration (FIG. 4).
2/Dose-Response Analysis of MLC1501 on the Expression of the Three Inflammatory Markers: IL-1β, IL-6, and TNF-α

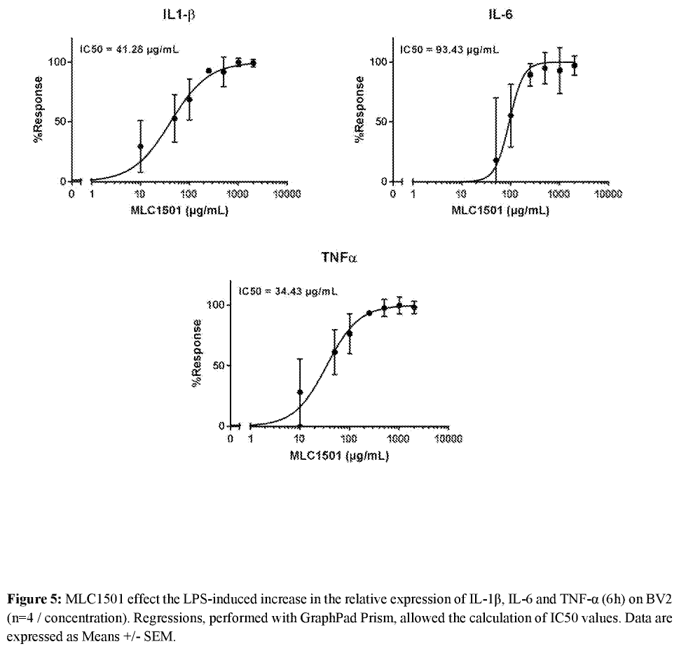

To perform the dose-response analysis, 8 different concentrations of MLC1501 were chosen: 0, 10, 50,100, 250, 500, 1000 and 2000 μg/mL. Administration of MLC1501 resulted in a total reduction in the relative over-expression of IL-1β, IL-6, and TNF-α in the presence of LPS in a dose-dependent manner. According to the regressions performed, the IC50 (Median Inhibitory Concentration) values for IL-1β, IL-6 and TNF-α are 41.28±1.50 g/mL, 93.43±2.61 μg/mL and 34.43±1.64 μg/mL respectively (FIG. 5).

### (b) MLC1501 Reduced the Relative Expression of Proinflammatory Genes in the In Vivo Focal Ischemia Model:

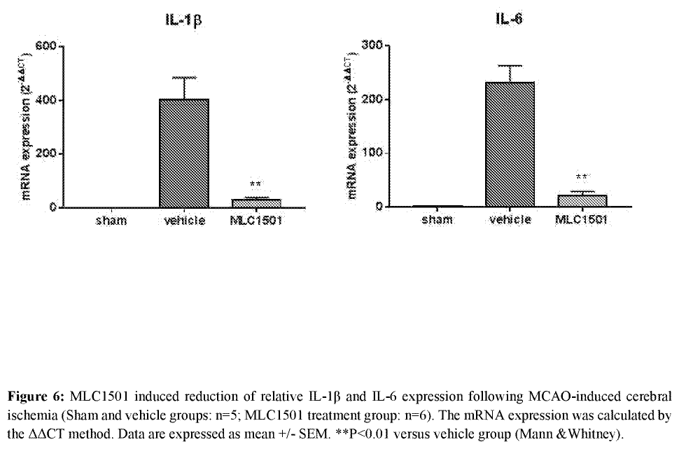

MCAO-induced focal ischemia in the vehicle group induced a high expression of proinflammatory genes. Administration of MLC1501 60 minutes before the onset of ischemia led to a significant reduction in the relative expression of genes encoding the cytokines IL-1 β and IL-6 (FIG. 6).

### 3) Conclusion

The study demonstrated that the administration of MLC1501 at the concentration of 250 ug/mL led to a decrease in inflammation by reducing the expression of mRNAs coding for genes involved in deleterious inflammation: IL-1β, IL-6, TNF-α, NLRP3 and iNOS. This phenomenon was observed both in vitro following LPS stimulation and in vivo following focal cerebral ischemia.

All references, articles, publications, patents, patent publications, and patent applications cited herein are incorporated by reference in their entireties for all purposes. However, mention of any reference, article, publication, patent, patent publication, and patent application herein are not, and should not, be taken as acknowledgment or any form of suggestion that they constitute valid prior art or form part of the common general knowledge in any country in the world.

1-60. (canceled)

61. A method of treating neuroinflammation in a subject, comprising administering to the subject an effective amount of a herbal composition comprising Radix Polygalae (root of thin leaf milkwort), Radix Astragali (root of membranous milkvetch), Rhizome Ligusticum chuanxiong and Radix Angelica senensis (root of Chinese angelica) or extracts thereof.

62. The method according to claim 61, wherein the herbal composition:
(a) further comprises Radix et Rhizome Salviae miltiorrhizae (red sage root), Radix Paeoniae rubra (red peony root), flower of Carthamus tinctorius (safflower), Semen persicae (Prunus persica seed) and Rhizome Acori tatarinowii (rhizome of grassleaf sweetflag) or extracts thereof;
(b) consists essentially of Radix Polygalae (root of thin leaf milkwort), Radix Astragali (root of membranous milkvetch), Rhizome Ligusticum chuanxiong and Radix Angelica senensis (root of Chinese angelica) or extracts thereof; or
(c) consists essentially of Radix Polygalae (root of thin leaf milkwort), Radix Astragali (root of membranous milkvetch), Rhizome Ligusticum chuanxiong, Radix Angelica senensis (root of Chinese angelica), Radix et Rhizome Salviae miltiorrhizae (red sage root), Radix Paeoniae rubra (red peony root), flower of Carthamus tinctorius (safflower), Semen persicae (Prunus persica seed) and Rhizome Acori tatarinowii (rhizome of grassleaf sweetflag) or extracts thereof.

63. The method according to claim 61, wherein the subject has diabetes mellitus.

64. The method according to claim 61, wherein the neuroinflammation is the result of an injury.

65. The method according to claim 61, wherein the neuroinflammation is the result of:
(a) an infection; or
(b) exposure to a toxin.

66. The method according to claim 61, wherein the neuroinflammation is associated with:
(a) a neurodegenerative disease;
(b) a neuropsychiatric condition;
(c) aging; or
(d) a disease or disorder selected from a group consisting of post-operative cognitive dysfunction, ischemia, brain tumor, chronic pain, headache, migraine, trigeminal neuralgia, cardiovascular disease and associated risk factors, a chronic peripheral inflammatory condition, gut microbiota perturbation, and herpes keratitis.

67. The method according to claim 61, wherein the neuroinflammation results from:
(a) a chemical intervention to treat an underlying condition or disease;
(b) a mechanical intervention to treat an underlying condition or disease;
(c) a surgical intervention to treat an underlying condition or disease.

68. The method according to claim 61, wherein the herbal composition is a pharmaceutical composition that:
(a) also comprises a pharmaceutically acceptable carrier or excipient;
(b) is the composition MLC901; or
(c) is the composition MLC1501.

69. The method of treatment according to claim 61, wherein an effective amount of the herbal composition is from about 1 mg/kg to about 100 mg/kg.

70. The method of according to claim 67, wherein the herbal composition is administered prior to, at the same time as, or following the administration of the intervention to treat an underlying condition or disease.

71. The method according to claim 61, wherein the subject is tested for, and confirmed as having neuroinflammation, prior to administering the herbal composition.

72. A method of preventing or limiting neuroinflammation in a subject at risk of neuroinflammation as a result of an insult, comprising administering to the subject an effective amount of a herbal composition comprising at least the four herbal components Radix Polygalae (root of thin leaf milkwort), Radix Astragali (root of membranous milkvetch), Rhizome Ligusticum chuanxiong and Radix Angelica senensis (root of Chinese angelica) or extracts thereof.

73. The method according to claim 72, wherein the herbal composition:
(a) further comprises at least one herbal component selected from the group consisting of Radix et Rhizome Salviae miltiorrhizae (red sage root), Radix Paeoniae rubra (red peony root), flower of Carthamus tinctorius (safflower), Semen persicae (Prunus persica seed) and Rhizome Acori tatarinowii (rhizome of grassleaf sweetflag) or an extract thereof;
(b) consists essentially of at least the four herbal components Radix Polygalae (root of thin leaf milkwort), Radix Astragali (root of membranous milkvetch), Rhizome Ligusticum chuanxiong and Radix Angelica senensis (root of Chinese angelica) or extracts thereof; or
(c) consists essentially of at least the nine herbal components Radix Polygalae (root of thin leaf milkwort), Radix Astragali (root of membranous milkvetch), Rhizome Ligusticum chuanxiong, Radix Angelica senensis (root of Chinese angelica), Radix et Rhizome Salviae miltiorrhizae (red sage root), Radix Paeoniae rubra (red peony root), flower of Carthamus tinctorius (safflower), Semen persicae (Prunus persica seed) and Rhizome Acori tatarinowii (rhizome of grassleaf sweetflag) or extracts thereof.

74. The method according to claim 72, wherein the herbal composition is a pharmaceutical composition that:
(a) also comprises a pharmaceutically acceptable carrier or excipient;
(b) is the composition MLC901; or
(c) is the composition MLC1501.

75. The method according to claim 64, wherein the injury is selected from the group consisting of spinal cord injury, stroke, traumatic brain injury, subarachnoid hemorrhage, subdural hemorrhage or extradural hemorrhage.

76. A method of treating a subject at risk of developing a disease or condition as a result of neuroinflammation, comprising administering to the subject an effective amount of a herbal composition comprising at least the four herbal components Radix Polygalae (root of thin leaf milkwort), Radix Astragali (root of membranous milkvetch), Rhizome Ligusticum chuanxiong and Radix Angelica senensis (root of Chinese angelica) or extracts thereof to prevent or mitigate the development of the disease or condition.

77. The method according to claim 76, wherein the herbal composition:
(a) further comprises at least one herbal component selected from the group consisting of Radix et Rhizome Salviae miltiorrhizae (red sage root), Radix Paeoniae rubra (red peony root), flower of Carthamus tinctorius (safflower), Semen persicae (Prunus persica seed) and Rhizome Acori tatarinowii (rhizome of grassleaf sweetflag) or an extract thereof;
(b) consists essentially of at least the four herbal components Radix Polygalae (root of thin leaf milkwort), Radix Astragali (root of membranous milkvetch), Rhizome Ligusticum chuanxiong and Radix Angelica senensis (root of Chinese angelica) or extracts thereof; or
(c) consists essentially of at least the nine herbal components Radix Polygalae (root of thin leaf milkwort), Radix Astragali (root of membranous milkvetch), Rhizome Ligusticum chuanxiong, Radix Angelica senensis (root of Chinese angelica), Radix et Rhizome Salviae miltiorrhizae (red sage root), Radix Paeoniae rubra (red peony root), flower of Carthamus tinctorius (safflower), Semen persicae (Prunus persica seed) and Rhizome Acori tatarinowii (rhizome of grassleaf sweetflag) or extracts thereof.

78. The method according to claim 76, wherein the herbal composition is a pharmaceutical composition that:
(a) also comprises a pharmaceutically acceptable carrier or excipient;
(b) is the composition MLC901; or
(c) is the composition MLC1501.

79. The method according to claim 61, comprising administering to the subject an effective amount of the herbal composition prior to an insult.

80. The method of claim 79, wherein the herbal composition is the composition MLC901 or the composition MLC1501.
